(19) United States
(12) Patent Application Publication
(10) Pub. No.: US 2024/0327343 A1
(43) Pub. Date: Oct. 3, 2024

(54) PREPARATION METHOD OF 3- (2, 2, 2-TRIFLUOROETHYL) PYRROLIDINE HYDROCHLORIDE

(71) Applicant: Kinnate Biopharma Inc., San Diego, CA (US)

(72) Inventors: Guanjun ZHANG, Xi'an (CN); Baokui SHUAI, Xi'an (CN); Yuqing SHAN, Xi'an (CN)

(21) Appl. No.: 18/580,519

(22) PCT Filed: Jul. 18, 2022
(86) PCT No.: PCT/CN2022/106174

§ 371 Date: Jan. 18, 2024

(30) Foreign Application Priority Data
Jul. 19, 2021 (CN) 202110813487.5
Jul. 19, 2021 (CN) 202110814726.9

(51) Int. Cl. C07D 207/08

(52) U.S. Cl. CPC C07D 207/08

(57) ABSTRACT

The present invention discloses a preparation method of 3-(2, 2, 2-trifluoroethyl) pyrrolidine hydrochloride and(S)-3-(2, 2, 2-trifluoroethyl) pyrrolidine hydrochloride.

### TECHNICAL FIELD

The present invention relates to the technical field of compound synthesis, in particular to a preparation method of 3-(2,2,2-trifluoroethyl) pyrrolidine hydrochloride an(S)-3-(2,2,2-trifluoroethyl) pyrrolidine hydrochloride.

### BACKGROUND OF THE INVENTION

3-(2,2,2-trifluoroethyl) pyrrolidine is an important intermediate compound. A literature, Journal of Organic Chemistry, 2019, p.16105-16115 (10.1021/acs.joc.9b02596), discloses a synthesis route as shown in FIG. 2, points out the disadvantages of a preparation method using expensive 3,3,3-trifluoropropionaldehyde as a raw material in a patent WO2013/53725 under UCB PHARMA GMBH, Belgium, and proposes a preparation method of using pyrrolidine-3-formic acid as a raw material and sulfur tetrafluoride as a fluorination reagent. However, the fluorination reagent, sulfur tetrafluoride, used in this method is a poisonous gas having a melting point of −121.5-−120.5° C. (lit.), a boiling point of −40.4° C. (lit.), and a pressure of 140 psi (21° C.). Thus, a tetrafluoroethylene container under an ultra-low temperature is required in a process using such reagent, which limits the scale-up application of this method.

### SUMMARY OF THE INVENTION

The present invention discloses a preparation method of 3-(2,2,2-trifluoroethyl) pyrrolidine hydrochloride, which comprises: reacting N-benzylpyrrolidone with trifluoroacetic anhydride to obtain 3-(1-chloro-2,2,2-trifluoroethylidene)-N-benzylpyrrole; dissolving 3-(1-chloro-2,2,2-trifluoroethylidene)-N-benzylpyrrole in methanol, and carrying out a substitution reaction in the presence of a catalyst and hydrogen to obtain 3-(2,2,2-trifluoroethyl)-N-benzylpyrrolidone; reacting 3-(2,2,2-trifluoroethyl)-N-benzylpyrrolidone with borane, and then adding methanol into the reaction mixture for further reaction to obtain 3-(2,2,2-trifluoroethyl)-N-benzylpyrrolidine; and dissolving 3-(2,2,2-trifluoroethyl)-N-benzylpyrrolidine in methanol, and carrying out a reaction in the presence of a catalyst and hydrogen to obtain the 3-(2,2,2-trifluoroethyl) pyrrolidine hydrochloride. By defining specific processes and parameters in the preparation method, the 3-(2,2,2-trifluoroethyl) pyrrolidine is obtained with high yield and purity.

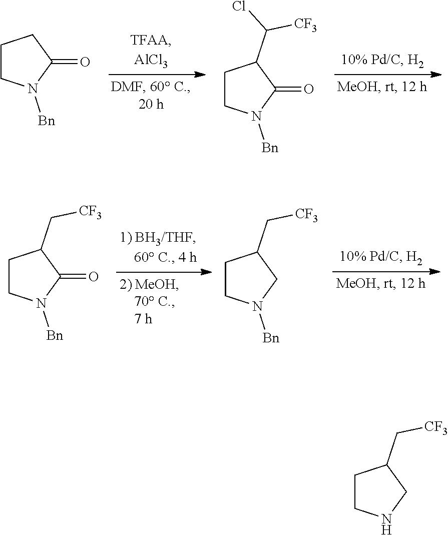 [SMILES: O=C1CCCN1Cc1ccccc1 ; O=C1C(C(Cl)C(F)(F)F)CCN1Cc1ccccc1 ; O=C1C(CC(F)(F)F)CCN1Cc1ccccc1 ; FC(F)(F)CC1CCN(Cc2ccccc2)C1 ; FC(F)(F)CC1CCNC1]

The present invention discloses a preparation method of(S)-3-(2,2,2-trifluoroethyl) pyrrolidine hydrochloride, which comprises: reacting R-naphthylethylamine with 4-chlorobutyryl chloride to obtain a product 1, reacting the product 1 with trifluoroacetic anhydride to obtain a product 2, dissolving the product 2 in methanol, and carrying out a substitution reaction in the presence of a catalyst and hydrogen to obtain a product 3, reacting the product 3 with borane, and adding methanol into the reaction mixture for further reaction to obtain a product 4, and dissolving the product 4 in methanol and carrying out a reaction in the presence of a catalyst and hydrogen to obtain the(S)-3-(2,2,2-trifluoroethyl) pyrrolidine hydrochloride. By defining specific processes and parameters in the preparation method, the(S)-3-(2,2,2-trifluoroethyl) pyrrolidine hydrochloride is obtained with high yield and purity.

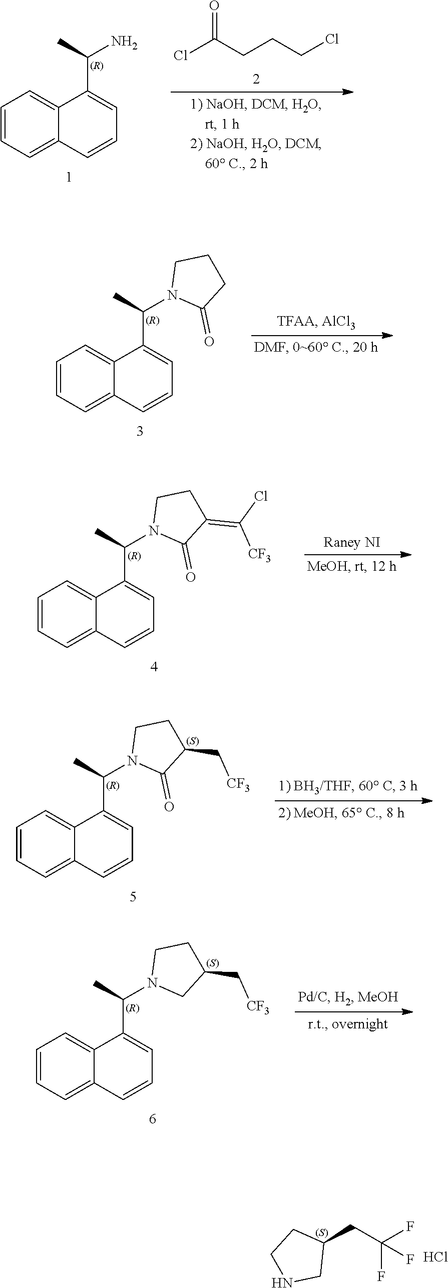

The object of the present invention is to provide a preparation method of 3-(2,2,2-trifluoroethyl) pyrrolidine hydrochloride, which uses easily available raw materials, and produces a product with high purity and yield.

In order to achieve the above object of the present invention, the following technical solutions are adopted:

A first aspect of the present invention provides a preparation method of 3-(2,2,2-trifluoroethyl) pyrrolidine hydrochloride, which comprises the following steps:
(a) reacting N-benzylpyrrolidone with trifluoroacetic anhydride to obtain 3-(1-chloro-2,2,2-trifluoroethylidene)-N-benzylpyrrole;
(b) dissolving 3-(1-chloro-2,2,2-trifluoroethylidene)-N-benzylpyrrole in methanol, and carrying out a substitution reaction in the presence of a catalyst and hydrogen to obtain 3-(2,2,2-trifluoroethyl)-N-benzylpyrrolidone;
(c) reacting 3-(2,2,2-trifluoroethyl)-N-benzylpyrrolidone with borane, and adding methanol into the reaction mixture for further reaction to obtain 3-(2,2,2-trifluoroethyl)-N-benzylpyrrolidine; and
(d) dissolving 3-(2,2,2-trifluoroethyl)-N-benzylpyrrolidine in methanol, and carrying out a reaction in the presence of a catalyst and hydrogen, filtering the reaction mixture after the reaction, collecting a filtrate, adding a solution of hydrogen chloride in dioxane to the filtrate, and concentrating the solvent to obtain a crude product, then dissolving the crude product in water, performing extraction with ethyl acetate, and freeze-drying the aqueous phase to obtain the 3-(2,2,2-trifluoroethyl) pyrrolidine hydrochloride.

Preferably, in the step (a), reacting N-benzylpyrrolidone with trifluoroacetic anhydride specifically comprises:
dissolving N-benzylpyrrolidone in N,N-dimethylformamide and cooling the mixture to 3-8° C., and then adding trifluoroacetic anhydride and aluminum trichloride successively for reaction.

Preferably, in the step (a), the mass ratio of N-benzylpyrrolidone, N,N-dimethylformamide, trifluoroacetic anhydride and aluminum trichloride is 1: (1-20): (1-4): (1-3); and
the reaction temperature is 55-65° C., and the reaction time is 12-40 hours.

Preferably, the step (a) further comprises subjecting a mixture obtained after the reaction to extraction and column chromatography, wherein a solvent used for the extraction is ethyl acetate, and developing agents used for the column chromatography are PE and EA with a volume ratio of 10:1.

Preferably, in the step (b), the mass ratio of 3-(1-chloro-2,2,2-trifluoroethylidene)-N-benzylpyrrole, methanol and the catalyst is 1: (1-20): (0.01-0.50); and the catalyst is 10% Pd/C.

Preferably, in the step (b), the conditions for the substitution reaction are as follows:
a reaction temperature at room temperature and a reaction time of 10-15 hours;
preferably, the step (b) further comprises: filtering a mixture obtained after the reaction, collecting a filtrate, concentrating the filtrate, then performing extraction with ethyl acetate for 2-4 times, then washing the organic phase with a saturated sodium bicarbonate aqueous solution until it is neutral, combining the organic phases, drying over anhydrous sodium sulfate, filtering, and concentrating the solvent to obtain the 3-(2,2,2-trifluoroethyl)-N-benzylpyrrolidone.

Preferably, in the step (c), reacting 3-(2,2,2-trifluoroethyl)-N-benzylpyrrolidone with borane specifically comprises:
dissolving 3-(2,2,2-trifluoroethyl)-N-benzylpyrrolidone in anhydrous tetrahydrofuran and cooling the mixture to 0-4° C., and then adding a solution of borane in tetrahydrofuran for reaction, wherein the mass ratio of 3-(2,2,2-trifluoroethyl)-N-benzylpyrrolidone, anhydrous tetrahydrofuran and the solution of borane in tetrahydrofuran is 1: (1-20): (2-10); the concentration of the solution of borane in tetrahydrofuran is 0.8-1.2 mol/L; the reaction temperature is 30-65° C., and the reaction time is 3.5-4.5 hours.

Preferably, in the step (c), adding methanol into the reaction mixture for further reaction specifically comprises:
after cooling the reaction mixture, adding methanol with a volume that is 0.1-10 times the volume of the reaction mixture for quenching, concentrating the solvent, and then adding methanol with a volume that is 1-10 times the volume of the reaction mixture for reaction, wherein the reaction temperature is 65-75° C. and the reaction time is 1-8 hours;
preferably, the step (c) further comprises: concentrating the mixture obtained after the reaction, then dissolving it in an aqueous hydrochloric acid solution and performing extraction with ethyl acetate, discarding the organic phase, adjusting the aqueous phase to be alkaline, and performing extraction with ethyl acetate for another 2-4 times, then combining the organic phases, drying over anhydrous sodium sulfate, filtering, and concentrating the solvent to obtain the 3-(2,2,2-trifluoroethyl)-N-benzylpyrrolidine; wherein the concentration of the aqueous hydrochloric acid solution is 3-5 mol/L.

Preferably, in the step (d), the mass ratio of 3-(2,2,2-trifluoroethyl)-N-benzylpyrrolidine, methanol and the catalyst is 1: (1-20): (0.01-0.50); and the catalyst is 10% Pd/C.

Preferably, in the step (d), the reaction conditions are as follows: a reaction temperature at room temperature and a reaction time of 10-30 hours;
preferably, the mass ratio of the solution of hydrogen chloride in dioxane to the filtrate is 1:0.10-2; and the concentration of the solution of hydrogen chloride in dioxane is 3.5-4.5 mol/L.

Compared with the prior art, the present invention comprises at least the following beneficial effects:
the preparation method of the present invention uses N-benzylpyrrolidone as a raw material to prepare 3-(2,2,2-trifluoroethyl) pyrrolidine hydrochloride. By defining specific processes and parameters in the preparation method, the 3-(2,2,2-trifluoroethyl) pyrrolidine is obtained with high yield and purity. Moreover, the preparation method has simple operation and can realize mass production.

The object of the present invention is to provide a preparation method of(S)-3-(2,2,2-trifluoroethyl) pyrrolidine hydrochloride, which uses easily available raw materials, and produces a product with high purity and yield.

In order to achieve the above object of the present invention, the following technical solutions are adopted:

A first aspect of the present invention provides a preparation method of(S)-3-(2,2,2-trifluoroethyl) pyrrolidine hydrochloride, which comprises the following steps:
(a) reacting R-naphthylethylamine with 4-chlorobutyryl chloride to obtain N—(R)-(1-(naphthalen-1-yl)ethyl) pyrrolidone;
(b) reacting N—(R)-(1-(naphthalen-1-yl)ethyl) pyrrolidone with trifluoroacetic anhydride to obtain 3-(1-chloro-2,2,2-trifluoroethylidene)-N—((R)-1-(naphthalen-1-yl)ethyl) pyrrolidone;
(c) dissolving 3-(2,2,2-trifluoroethylidene)-N—((R)-1-(naphthalen-1-yl)ethyl) pyrrolidone in methanol, and carrying out a substitution reaction in the presence of a catalyst and hydrogen to obtain(S)-3-(2,2,2-trifluoroethyl)-N—((R)-1-(naphthalen-1-yl)ethyl) pyrrolidone;
(d) reacting(S)-3-(2,2,2-trifluoroethyl)-N—((R)-1-(naphthalen-1-yl)ethyl) pyrrolidone with borane, and adding methanol into the reaction mixture for further reaction to obtain(S)-3-(2,2,2-trifluoroethyl)-N—((R)-1-(naphthalen-1-yl)ethyl) pyrrolidine; and
(e) dissolving(S)-3-(2,2,2-trifluoroethyl)-N—((R)-1-(naphthalen-1-yl)ethyl) pyrrolidine in methanol and carrying out a reaction in the presence of a catalyst and hydrogen, filtering the reaction mixture after the reaction, collecting a filtrate, adding a solution of hydrogen chloride in dioxane to the filtrate, and concentrating the solvent to obtain a crude product, then dissolving the crude product in water, performing extraction with ethyl acetate, and freeze-drying the aqueous phase to obtain the(S)-3-(2,2,2-trifluoroethyl) pyrrolidine hydrochloride.

Preferably, in the step (a), reacting R-naphthylethylamine with 4-chlorobutyryl chloride specifically comprises:
dissolving R-naphthylethylamine in dichloromethane, adding an aqueous sodium hydroxide solution, stirring vigorously, adding dropwise a solution of 4-chlorobutyryl chloride in dichloromethane, reacting at room temperature for 1 hour, and then raising the temperature of the reaction solution to 55-65° C. for further reacting for 2-4 hours, or reacting at room temperature for 20-30 hours.

Preferably, in the step (a), the mass ratio of R-naphthylethylamine, dichloromethane, the aqueous sodium hydroxide solution and the solution of 4-chlorobutyryl chloride in dichloromethane is 1: (5-10): (0.6-2.8): (0.4-4), wherein the concentration of the aqueous sodium hydroxide solution is 50% and the concentration of the solution of 4-chlorobutyryl chloride in dichloromethane is 50%.

Preferably, in the step (b), reacting N—(R)-(1-(naphthalen-1-yl)ethyl) pyrrolidone with trifluoroacetic anhydride specifically comprises:
dissolving N—(R)-(1-(naphthalen-1-yl)ethyl) pyrrolidone in N,N-dimethylformamide and cooling the mixture to 3-8° C., and then adding trifluoroacetic anhydride and aluminum trichloride successively for reaction.

Preferably, in the step (b), the mass ratio of N—(R)-(1-(naphthalen-1-yl)ethyl) pyrrolidone, N,N-dimethylformamide, trifluoroacetic anhydride and aluminum trichloride is 1: (1-20): (1-4): (0.3-3); and the reaction temperature is 55-65° C., and the reaction time is 12-40 hours.

Preferably, the step (b) further comprises subjecting a mixture obtained after the reaction to extraction and column chromatography, wherein a solvent used for the extraction is ethyl acetate, and developing agents used for the column chromatography are PE and EA with a volume ratio of 10:1.

Preferably, in the step (c), the mass ratio of 3-(1-chloro-2,2,2-trifluoroethylidene)-N—((R)-1-(naphthalen-1-yl)ethyl) pyrrolidone, methanol and the catalyst is 1: (1-50): (0.1-1); and
the catalyst is Raney nickel.

Preferably, in the step (c), the conditions for the substitution reaction are as follows:
a reaction temperature at room temperature and a reaction time of 20-24 hours.

Preferably, the step (c) further comprises: filtering a mixture obtained after the reaction, collecting a filtrate, concentrating the filtrate, then performing extraction with ethyl acetate for 2-4 times, combining the organic phases, drying over anhydrous sodium sulfate, filtering, and concentrating the solvent to obtain the(S)-3-(2,2,2-trifluoroethyl)-N—((R)-1-(naphthalen-1-yl)ethyl) pyrrolidone.

Preferably, in the step (d), reacting(S)-3-(2,2,2-trifluoroethyl)-N—((R)-1-(naphthalen-1-yl)ethyl) pyrrolidone with borane specifically comprises:
dissolving(S)-3-(2,2,2-trifluoroethyl)-N—((R)-1-(naphthalen-1-yl)ethyl) pyrrolidone in anhydrous tetrahydrofuran and cooling the mixture to 0-4° C., and then adding a solution of borane in tetrahydrofuran for reaction, wherein the mass ratio of(S)-3-(2,2,2-trifluoroethyl)-N—((R)-1-(naphthalen-1-yl)ethyl) pyrrolidone, anhydrous tetrahydrofuran and the solution of borane in tetrahydrofuran is 1: (1-20): (2-10); the concentration of the solution of borane in tetrahydrofuran is 0.8-1.2 mol/L; the reaction temperature is 30-65° C., and the reaction time is 3.5-4.5 hours.

Preferably, in the step (d), adding methanol into the reaction mixture for further reaction specifically comprises:
after cooling the reaction mixture, adding methanol with a volume that is 0.1-10 times the volume of the reaction mixture for quenching, concentrating the solvent, and then adding methanol with a volume that is 1-10 times the volume of the reaction mixture for reaction, wherein the reaction temperature is 65-75° C. and the reaction time is 1-8 hours;
preferably, the step (d) further comprises: concentrating the mixture obtained after the reaction, then dissolving it in an aqueous hydrochloric acid solution, performing extraction with ethyl acetate, discarding the organic phase, adjusting the aqueous phase to be alkaline, and performing extraction with ethyl acetate for another 2-4 times, then combining the organic phases, drying over anhydrous sodium sulfate, filtering, and concentrating the solvent to obtain the(S)-3-(2,2,2-trifluoroethyl)-N—((R)-1-(naphthalen-1-yl)ethyl) pyrrolidine; wherein the concentration of the aqueous hydrochloric acid solution is 3-5 mol/L.

Preferably, in the step (e), the mass ratio of(S)-3-(2,2,2-trifluoroethyl)-N—((R)-1-(naphthalen-1-yl)ethyl) pyrrolidine, methanol and the catalyst is 1: (1-20): (0.01-0.50); and
the catalyst is 10% Pd/C.

Preferably, in the step (e), the reaction conditions are as follows: a reaction temperature at room temperature and a reaction time of 10-30 hours.

Preferably, in the step (e), the mass ratio of the solution of hydrogen chloride in dioxane to the filtrate is 1:(0.10-2); and the concentration of the solution of hydrogen chloride in dioxane is 3.5-4.5 mol/L.

Compared with the prior art, the present invention comprises at least the following beneficial effects:
the preparation method of the present invention uses R-naphthylethylamine as a raw material to prepare(S)-3-(2,2,2-trifluoroethyl) pyrrolidine hydrochloride. By defining specific processes and parameters in the preparation method, the(S)-3-(2,2,2-trifluoroethyl) pyrrolidine hydrochloride is obtained with high yield and purity. Moreover, the preparation method has simple operation and can realize mass production.

### BRIEF DESCRIPTION OF THE DRAWINGS

In order to illustrate the technical solutions in particular embodiments of the present invention or in the prior art more clearly, accompanying drawings to be used in the description of the particular embodiments or the prior art will be briefly introduced below. In all the drawings, similar elements or parts are generally identified by similar reference numerals. In the drawings, each element or part is not necessarily drawn to the actual scale.

FIG. 1 is a synthetic procedure of the 3-(2,2,2-trifluoroethyl) pyrrolidine hydrochloride according to an embodiment of the present invention; and

FIG. 2 is a synthetic procedure of the prior art provided in the background art of the present invention.

FIG. 3 is a synthetic procedure of the(S)-3-(2,2,2-trifluoroethyl) pyrrolidine hydrochloride according to an embodiment of the present invention; and

FIG. 4 is a synthetic procedure of the prior art provided in the background art of the present invention.

### DETAILED DESCRIPTION OF THE INVENTION

The embodiments of the technical solutions of the present invention will be described in detail below in combination with examples. The following examples are only used to more clearly illustrate the technical solutions of the present invention, therefore are illustrative and non-limiting to the protection scope of the present invention.

It should be noted that, unless otherwise specified, the technical terms or scientific terms used in present application shall have the general meanings as understood by those skilled in the art to which the present invention belongs.

### Example 1

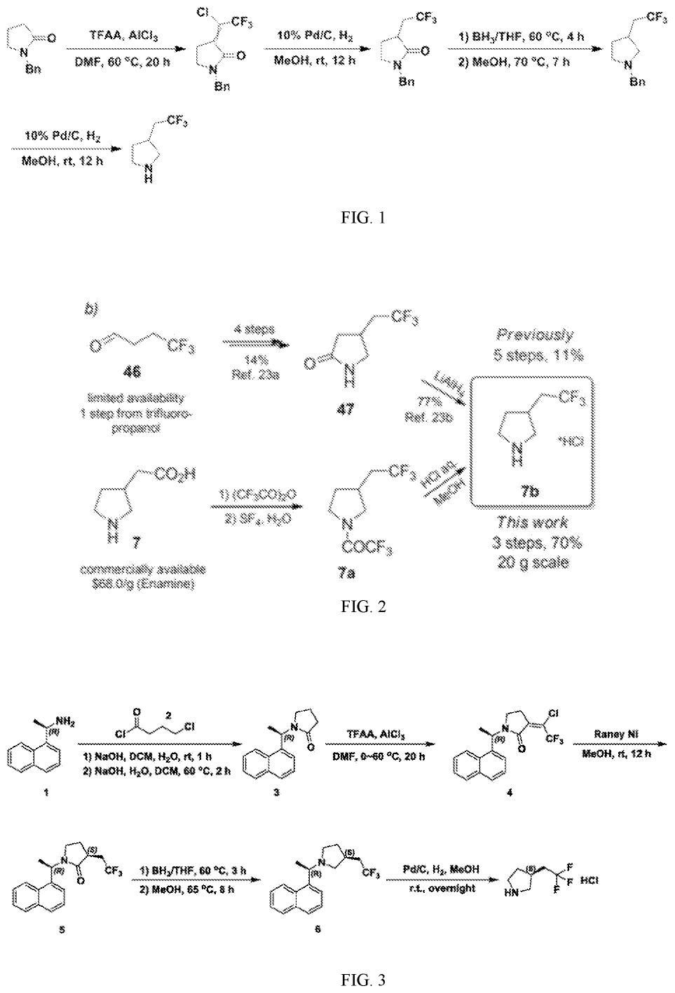

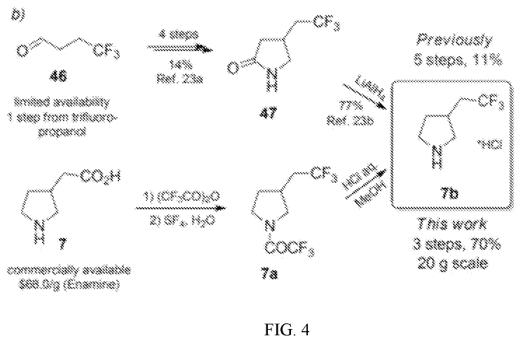

This Example is a preparation method of 3-(2,2,2-trifluoroethyl) pyrrolidine hydrochloride. As shown in FIG. 1, the preparation method comprises the following steps:
(a) dissolving N-benzylpyrrolidone in N,N-dimethylformamide and cooling the mixture to 4° C., then adding trifluoroacetic anhydride, slowly adding aluminum trichloride and stirring until it was completely dissolved to obtain a mixed solution, raising the temperature of the mixed solution to 60° C. for reaction for 20 hours, cooling the reaction solution to room temperature, adding water to quench the reaction, performing extraction with ethyl acetate for 3 times, combining the organic phases, drying over anhydrous sodium sulfate, filtering, concentrating the solvent, and performing column chromatography (petroleum ether/ethyl acetate=10:1) to obtain the product 3-(1-chloro-2,2,2-trifluoroethylidene)-N-benzylpyrrole as a blackish oil,
wherein, the mass ratio of N-benzylpyrrolidone, N,N-dimethylformamide, trifluoroacetic anhydride and aluminum trichloride was 1:4.49:3.60:1.14;
(b) dissolving 3-(1-chloro-2,2,2-trifluoroethylidene)-N-benzylpyrrole in methanol and adding 10% Pd/C as a catalyst, then reacting at room temperature under hydrogen for 12 hours, filtering the reaction solution, collecting a filtrate, concentrating the filtrate, performing extraction with ethyl acetate for 3 times, washing the product to neutral with saturated sodium bicarbonate, combining the organic phases, drying over anhydrous sodium sulfate, filtering, and concentrating the solvent to obtain the product 3-(2,2,2-trifluoroethyl)-N-benzylpyrrolidone as a light yellow oil,
wherein, the mass ratio of 3-(1-chloro-2,2,2-trifluoroethylidene)-N-benzylpyrrole, methanol and 10% Pd/C catalyst was 1:5.17:0.10;
(c) dissolving 3-(2,2,2-trifluoroethyl)-N-benzylpyrrolidone in anhydrous tetrahydrofuran and cooling the mixture to 2° C., then adding a solution of borane in tetrahydrofuran and reacting at 60° C. for 4 hours, cooling the reaction solution to room temperature, adding methanol with a volume that was 5 times the volume of the reaction solution for quenching the reaction, concentrating the solvent, adding methanol with a volume that was 5 times the volume of the reaction solution again and reacting at 70° C. for 7 hours, then rotary evaporating the reaction solution to dryness and dissolving in 4 mol/L aqueous hydrochloric acid solution, performing back-extraction with ethyl acetate, discarding the organic phase, adjusting the aqueous phase to be alkaline with 6 mol/L aqueous sodium hydroxide solution, performing extraction with ethyl acetate, drying the organic phase over anhydrous sodium sulfate, filtering, and concentrating the solvent to obtain the product 3-(2,2,2-trifluoroethyl)-N-benzylpyrrolidine as a light yellow oil;
wherein, the mass ratio of 3-(2,2,2-trifluoroethyl)-N-benzylpyrrolidone, anhydrous tetrahydrofuran and the solution of borane in tetrahydrofuran was 1:1.40:6.99; and the concentration of the solution of borane in tetrahydrofuran was 1 mol/L;
(d) dissolving 3-(2,2,2-trifluoroethyl)-N-benzylpyrrolidine in methanol and adding 10% Pd/C as a catalyst, then reacting at room temperature under hydrogen for 12 hours, filtering the reaction solution, collecting a filtrate, adding 4 mol/L solution of hydrogen chloride in dioxane to the filtrate, and concentrating the solvent to obtain a crude product, then dissolving the crude product in water and performing extraction with ethyl acetate, discarding the organic phase and freeze-drying the aqueous phase to obtain the 3-(2,2,2-trifluoroethyl) pyrrolidine hydrochloride;
wherein, the mass ratio of 3-(2,2,2-trifluoroethyl)-N-benzylpyrrolidine, methanol, and 10% Pd/C catalyst was 1:10.8:0.11.

### Example 2

This Example is a preparation method of 3-(2,2,2-trifluoroethyl) pyrrolidine hydrochloride, which comprises the following steps:
(a) dissolving N-benzylpyrrolidone in N,N-dimethylformamide and cooling the mixture to 4° C., adding trifluoroacetic anhydride, slowly adding aluminum trichloride and stirring until it was completely dissolved to obtain a mixed solution, raising the temperature of the mixed solution to 60° C. for reaction for 12 hours, cooling the reaction solution to room temperature, adding water to quench the reaction, performing extraction with ethyl acetate for 3 times, combining the organic phases, drying over anhydrous sodium sulfate, filtering, concentrating the solvent, and performing column chromatography (petroleum ether/ethyl acetate=10:1) to obtain the product 3-(1-chloro-2,2,2-trifluoroethylidene)-N-benzylpyrrole as a blackish oil,
wherein, the mass ratio of N-benzylpyrrolidone, N,N-dimethylformamide, trifluoroacetic anhydride and aluminum trichloride was 1:1.43:1.06:2.7;
(b) dissolving 3-(1-chloro-2,2,2-trifluoroethylidene)-N-benzylpyrrole in methanol and adding 10% Pd/C as a catalyst, then reacting at room temperature under hydrogen for 8 hours, filtering the reaction solution, collecting a filtrate, concentrating the filtrate and dissolving in ethyl acetate, washing the product to neutral with saturated sodium bicarbonate, combining the organic phases, drying over anhydrous sodium sulfate, filtering, and concentrating the solvent to obtain the product 3-(2,2,2-trifluoroethyl)-N-benzylpyrrolidone as a light yellow oil,
wherein, the mass ratio of 3-(1-chloro-2,2,2-trifluoroethylidene)-N-benzylpyrrole, methanol and 10% Pd/C catalyst was 1:16.38:0.50;
(c) dissolving 3-(2,2,2-trifluoroethyl)-N-benzylpyrrolidone in anhydrous tetrahydrofuran and cooling the mixture to 2° C., then adding a solution of borane in tetrahydrofuran and reacting at 30° C. for 4 hours, cooling the reaction solution to room temperature, adding methanol with a volume that was 5 times the volume of the reaction solution for quenching the reaction, concentrating the solvent, adding methanol with a volume that was 5 times the volume of the reaction solution again and reacting at 70° C. for 2 hours, then rotary evaporating the reaction solution to dryness and dissolving in 4 mol/L aqueous hydrochloric acid solution, performing back-extraction with ethyl acetate, discarding the organic phase, adjusting the aqueous phase to be alkaline with 6 mol/L aqueous sodium hydroxide solution, performing extraction with ethyl acetate for another 3 times, combining organic phases, drying over anhydrous sodium sulfate, filtering, and concentrating the solvent to obtain the product 3-(2,2,2-trifluoroethyl)-N-benzylpyrrolidine as a light yellow oil,
wherein, the mass ratio of 3-(2,2,2-trifluoroethyl)-N-benzylpyrrolidone, anhydrous tetrahydrofuran and the solution of borane in tetrahydrofuran was 1:10.20:2.85; and the concentration of the solution of borane in tetrahydrofuran was 1 mol/L;
(d) dissolving 3-(2,2,2-trifluoroethyl)-N-benzylpyrrolidine in methanol and adding 10% Pd/C as a catalyst, then reacting at room temperature under hydrogen for 30 hours, filtering the reaction solution, collecting a filtrate, adding 4 mol/L solution of hydrogen chloride in dioxane to the filtrate, concentrating the solvent, dissolving the crude product in water and performing extraction with ethyl acetate, discarding the organic phase and freeze-drying the aqueous phase to obtain the 3-(2,2,2-trifluoroethyl) pyrrolidine hydrochloride;
wherein, the mass ratio of 3-(2,2,2-trifluoroethyl)-N-benzylpyrrolidine, methanol and 10% Pd/C catalyst was 1:5.4:0.32.

### Example 3

This Example is a preparation method of 3-(2,2,2-trifluoroethyl) pyrrolidine, which comprises the following steps:
(a) dissolving N-benzylpyrrolidone in N,N-dimethylformamide and cooling the mixture to 4° C., adding trifluoroacetic anhydride, slowly adding aluminum trichloride and stirring until it was completely dissolved to obtain a mixed solution, raising the temperature of the mixed solution to 60° C. for reaction for 20 hours, cooling the reaction solution to room temperature, adding water to quench the reaction, performing extraction with ethyl acetate for 3 times, combining the organic phases, drying over anhydrous sodium sulfate, filtering, concentrating the solvent, and performing column chromatography (petroleum ether/ethyl acetate=10:1) to obtain the product 3-(1-chloro-2,2,2-trifluoroethylidene)-N-benzylpyrrole as a blackish oil,
wherein, the mass ratio of N-benzylpyrrolidone, N,N-dimethylformamide, trifluoroacetic anhydride and aluminum trichloride was 1:9.48:2.4:1.14;
(b) dissolving 3-(1-chloro-2,2,2-trifluoroethylidene)-N-benzylpyrrole in methanol and adding 10% Pd/C as a catalyst, then reacting at room temperature under hydrogen for 12 hours, filtering the reaction solution, collecting a filtrate, concentrating the filtrate and dissolving in ethyl acetate, washing the product to neutral with saturated sodium bicarbonate, drying the organic phase over anhydrous sodium sulfate, filtering, and concentrating the solvent to obtain the product 3-(2,2,2-trifluoroethyl)-N-benzylpyrrolidone as a light yellow oil,
wherein, the mass ratio of 3-(1-chloro-2,2,2-trifluoroethylidene)-N-benzylpyrrole, methanol and 10% Pd/C catalyst was 1:7.91:0.05;
(c) dissolving 3-(2,2,2-trifluoroethyl)-N-benzylpyrrolidone in anhydrous tetrahydrofuran and cooling the mixture to 2° C., then adding a solution of borane in tetrahydrofuran and reacting at 60° C. for 4 hours, cooling the reaction solution to room temperature, adding methanol with a volume that was 5 times the volume of the reaction solution for quenching the reaction, concentrating the solvent, adding methanol with a volume that was 5 times the volume of the reaction solution again and reacting at 70° C. for 7 hours, then rotary evaporating the reaction solution to dryness and dissolving in 4 mol/L aqueous hydrochloric acid solution, performing back-extraction with ethyl acetate, discarding the organic phase, adjusting the aqueous phase to be alkaline with 6 mol/L aqueous sodium hydroxide solution, performing extraction with ethyl acetate for another 3 times, combining the organic phases, drying over anhydrous sodium sulfate, filtering, and concentrating the solvent to obtain the product 3-(2,2,2-trifluoroethyl)-N-benzylpyrrolidine as a light yellow oil.

Wherein, the mass ratio of 3-(2,2,2-trifluoroethyl)-N-benzylpyrrolidone, anhydrous tetrahydrofuran and the solution of borane in tetrahydrofuran was 1:4.45:4.00; and the concentration of the solution of borane in tetrahydrofuran was 1 mol/L;
(d) dissolving 3-(2,2,2-trifluoroethyl)-N-benzylpyrrolidine in methanol and adding 10% Pd/C as a catalyst, then reacting at room temperature under hydrogen for 12 hours, filtering the reaction solution, collecting a filtrate, adding 4 mol/L solution of hydrogen chloride in dioxane to the filtrate, and concentrating the solvent to obtain a crude product, then dissolving the crude product in water and performing extraction with ethyl acetate, discarding the organic phase and freeze-drying the aqueous phase to obtain the 3-(2,2,2-trifluoroethyl) pyrrolidine hydrochloride.

Wherein, the mass ratio of 3-(2,2,2-trifluoroethyl)-N-benzylpyrrolidine, methanol and 10% Pd/C catalyst was 1:15.8:0.05.

### Experimental Example

3-(2,2,2-trifluoroethyl) pyrrolidine hydrochloride was prepared according to examples 1-3, respectively; and the yield of the product prepared in each step of each example was calculated according to the following calculation method;
the calculation formula of the yield of reaction in each step is mₚ×Mₛ/(mₛ×Mₚ)×100%, where mₚ is the actual output of the product, Mₛ is the molar mass of the raw material, mₛ is the theoretical addition amount of the raw material, and Mₚ is the molar mass of the product.

The calculation results are as shown in Table 1:

TABLE 1

| | Exam- | Exam- | Exam- |
|---|---|---|---|
| Group/Yield | ple 1 | ple 2 | ple 3 |
| 3-(1-chloro-2,2,2-trifluoroethylidene)-N- | 76.7% | 66.5% | 75.5% |
| benzylpyrrole | | | |
| 3-(2,2,2-trifluoroethyl)-N-benzylpyrrolidone | 88.7% | 86.7% | 77.2% |
| 3-(2,2,2-trifluoroethyl)-N-benzylpyrrolidine | 52.2% | 17.8% | 43.9% |
| 3-(2,2,2-trifluoroethyl)pyrrolidine | 95.4% | 87.2% | 65.8% |
| hydrochloride | | | |

It can be seen from Table 1 that:

the preparation method of the present invention realizes the preparation of 3-(2,2,2-trifluoroethyl) pyrrolidine hydrochloride by using N-benzylpyrrolidone as a raw material. By defining specific processes and parameters in the preparation method, the 3-(2,2,2-trifluoroethyl) pyrrolidine is obtained with high yield and purity. Moreover, the preparation method has simple operation and can realize mass production.

### Example 4

This Example is a preparation method of(S)-3-(2,2,2-trifluoroethyl) pyrrolidine hydrochloride. As shown in FIG. 3, the preparation method comprises the following steps:
(a) dissolving R-naphthylethylamine in dichloromethane, adding an aqueous sodium hydroxide solution, stirring vigorously, adding dropwise a solution of 4-chlorobutyryl chloride in dichloromethane, reacting at room temperature for 1 hour, and then raising the temperature of the reaction solution to 60° C. for further reacting for 3 hours; after the reaction was completed, separating the liquids, extracting the aqueous phase twice with dichloromethane, combining the organic phases, drying over anhydrous sodium sulfate, filtering, and concentrating the solvent to obtain the product N—(R)-(1-(naphthalen-1-yl)ethyl) pyrrolidone as a yellow oil;
wherein: the mass ratio of R-naphthylethylamine, dichloromethane, the aqueous sodium hydroxide solution and the solution of 4-chlorobutyryl chloride in dichloromethane was 1:6:1.6:0.82, wherein the concentration of the aqueous sodium hydroxide solution was 50% and the concentration of the solution of 4-chlorobutyryl chloride in dichloromethane was 50%;
(b) dissolving N—(R)-(1-(naphthalen-1-yl)ethyl) pyrrolidone in N,N-dimethylformamide and cooling the mixture to 4° C., then adding trifluoroacetic anhydride, slowly adding aluminum trichloride and stirring until it was completely dissolved to obtain a mixed solution, raising the temperature of the mixed solution to 60° C. for reaction for 22 hours, cooling the reaction solution to room temperature, adding water to quench the reaction, performing extraction with ethyl acetate for 3 times, combining the organic phases, drying over anhydrous sodium sulfate, filtering, concentrating the solvent, and performing column chromatography (petroleum ether/ethyl acetate=10:1) to obtain the product 3-(1-chloro-2,2,2-trifluoroethylidene)-N—((R)-1-(naphthalen-1-yl)ethyl) pyrrolidone as a blackish oil,
wherein the mass ratio of (R)—N-(1-(naphthalen-1-yl)ethyl) pyrrolidone, N,N-dimethylformamide, trifluoroacetic anhydride and aluminum trichloride was 1:5.11:2.64:0.84;
(c) dissolving 3-(1-chloro-2,2,2-trifluoroethylidene)-N—((R)-1-(naphthalen-1-yl)ethyl) pyrrolidone in methanol and adding Raney nickel as a catalyst, then reacting at room temperature under hydrogen for 22 hours, filtering the reaction solution, collecting a filtrate, concentrating the filtrate, performing extraction with ethyl acetate for 3 times, washing the product to neutral with saturated sodium bicarbonate, combining the organic phases, drying over anhydrous sodium sulfate, filtering, and concentrating the solvent to obtain the product(S)-3-(2,2,2-trifluoroethyl)-N—((R)-1-(naphthalen-1-yl)ethyl) pyrrolidone as a colorless oil,
wherein the mass ratio of 3-(1-chloro-2,2,2-trifluoroethylidene)-N—((R)-1-(naphthalen-1-yl)ethyl) pyrrolidone, methanol and the catalyst Raney nickel was 1:10.0:0.40; and
(d) dissolving(S)-3-(2,2,2-trifluoroethyl)-N—((R)-1-(naphthalen-1-yl)ethyl) pyrrolidone in anhydrous tetrahydrofuran and cooling the mixture to 2° C., then adding a solution of borane in tetrahydrofuran and reacting at 60° C. for 4 hours, cooling the reaction solution to room temperature, adding methanol with a volume that was 5 times the volume of the reaction solution for quenching the reaction, concentrating the solvent, adding methanol with a volume that was 5 times the volume of the reaction solution again and reacting at 70° C. for 6 hours, then rotary evaporating the reaction solution to dryness and dissolving in 4 mol/L aqueous hydrochloric acid solution, performing back-extraction with ethyl acetate, discarding the organic phase, adjusting the aqueous phase to be alkaline with 6 mol/L aqueous sodium hydroxide solution, performing extraction with ethyl acetate, drying the organic phase over anhydrous sodium sulfate, filtering, and concentrating the solvent to obtain the product(S)-3-(2,2,2-trifluoroethyl)-N—((R)-1-(naphthalen-1-yl)ethyl) pyrrolidine as a light yellow oil;
wherein the mass ratio of(S)-3-(2,2,2-trifluoroethyl)-N—((R)-1-(naphthalen-1-yl)ethyl) pyrrolidone, anhydrous tetrahydrofuran and the solution of borane in tetrahydrofuran was 1:4.35:6.23; and the concentration of the solution of borane in tetrahydrofuran was 1 mol/L;
(e) dissolving(S)-3-(2,2,2-trifluoroethyl)-N—((R)-1-(naphthalen-1-yl)ethyl) pyrrolidine in methanol and adding 10% Pd/C as a catalyst, then reacting at room temperature under hydrogen for 20 hours, filtering the reaction solution, collecting a filtrate, adding 4 mol/L solution of hydrogen chloride in dioxane to the filtrate, and concentrating the solvent to obtain a crude product, then dissolving the crude product in water and performing extraction with ethyl acetate, discarding the organic phase and freeze-drying the aqueous phase to obtain the(S)-3-(2,2,2-trifluoroethyl) pyrrolidine hydrochloride;
wherein the mass ratio of(S)-3-(2,2,2-trifluoroethyl)-N—((R)-1-(naphthalen-1-yl)ethyl) pyrrolidine, methanol and 10% Pd/C catalyst was 1:10.8:0.11.

### Example 5

This Example is a preparation method of(S)-3-(2,2,2-trifluoroethyl) pyrrolidine hydrochloride. As shown in FIG. 3, the preparation method comprises the following steps:
(a) dissolving R-naphthylethylamine in dichloromethane, adding an aqueous sodium hydroxide solution, stirring vigorously, adding dropwise a solution of 4-chlorobutyryl chloride in dichloromethane, and reacting at room temperature for 24 hours. After the reaction was completed, separating the liquids, extracting the aqueous phase twice with dichloromethane, combining the organic phases, drying over anhydrous sodium sulfate, filtering, and concentrating the solvent to obtain the product N—(R)-(1-(naphthalen-1-yl)ethyl) pyrrolidone as a yellow oil;
wherein: the mass ratio of R-naphthylethylamine, dichloromethane, the aqueous sodium hydroxide solution and the solution of 4-chlorobutyryl chloride in dichloromethane was 1:10:0.8:0.8, wherein the concentration of the aqueous sodium hydroxide solution was 50% and the concentration of the solution of 4-chlorobutyryl chloride in dichloromethane was 50%;
(b) dissolving N—(R)-(1-(naphthalen-1-yl)ethyl) pyrrolidone in N,N-dimethylformamide and cooling the mixture to 4° C., adding trifluoroacetic anhydride, then slowly adding aluminum trichloride and stirring until it was completely dissolved to obtain a mixed solution, raising the temperature of the mixed solution to 65° C. for reaction for 15 hours, cooling the reaction solution to room temperature, adding water to quench the reaction, performing extraction with ethyl acetate for 3 times, combining the organic phases, drying over anhydrous sodium sulfate, filtering, concentrating the solvent, and performing column chromatography (petroleum ether/ethyl acetate=10:1) to obtain the product 3-(1-chloro-2,2,2-trifluoroethylidene)-N—((R)-1-(naphthalen-1-yl)ethyl) pyrrolidone as a blackish oil,
wherein the mass ratio of (R)—N-(1-(naphthalen-1-yl)ethyl) pyrrolidone, N,N-dimethylformamide, trifluoroacetic anhydride and aluminum trichloride was 1:10:1:2;
(c) dissolving 3-(1-chloro-2,2,2-trifluoroethylidene)-N—((R)-1-(naphthalen-1-yl)ethyl) pyrrolidone in methanol and adding Raney nickel as a catalyst, then reacting at room temperature under hydrogen for 24 hours, filtering the reaction solution, collecting a filtrate, concentrating the filtrate, performing extraction with ethyl acetate for 3 times, washing the product to neutral with saturated sodium bicarbonate, combining the organic phases, drying over anhydrous sodium sulfate, filtering, and concentrating the solvent to obtain the product(S)-3-(2,2,2-trifluoroethyl)-N—((R)-1-(naphthalen-1-yl)ethyl) pyrrolidone as a colorless oil,
wherein the mass ratio of 3-(1-chloro-2,2,2-trifluoroethylidene)-N—((R)-1-(naphthalen-1-yl)ethyl) pyrrolidone, methanol and the catalyst Raney nickel was 1:20:0.1;
(d) dissolving(S)-3-(2,2,2-trifluoroethyl)-N—((R)-1-(naphthalen-1-yl)ethyl) pyrrolidone in anhydrous tetrahydrofuran and cooling the mixture to 2° C., then adding a solution of borane in tetrahydrofuran and reacting at 30° C. for 4.5 hours, cooling the reaction solution to room temperature, adding methanol with a volume that was 5 times the volume of the reaction solution for quenching the reaction, concentrating the solvent, adding methanol with a volume that was 5 times the volume of the reaction solution again and reacting at 75° C. for 2 hours, then rotary evaporating the reaction solution to dryness and dissolving in 4 mol/L aqueous hydrochloric acid solution, performing back-extraction with ethyl acetate, discarding the organic phase, adjusting the aqueous phase to be alkaline with 6 mol/L aqueous sodium hydroxide solution, performing extraction with ethyl acetate, drying the organic phase over anhydrous sodium sulfate, filtering, and concentrating the solvent to obtain the product(S)-3-(2,2,2-trifluoroethyl)-N—((R)-1-(naphthalen-1-yl)ethyl) pyrrolidine as a light yellow oil;
wherein the mass ratio of(S)-3-(2,2,2-trifluoroethyl)-N—((R)-1-(naphthalen-1-yl)ethyl) pyrrolidone, anhydrous tetrahydrofuran and the solution of borane in tetrahydrofuran was 1:1:10; and the concentration of the solution of borane in tetrahydrofuran was 0.8 mol/L;
(e) dissolving(S)-3-(2,2,2-trifluoroethyl)-N—((R)-1-(naphthalen-1-yl)ethyl) pyrrolidine in methanol and adding 10% Pd/C as a catalyst, then reacting at room temperature under hydrogen for 25 hours, filtering the reaction solution, collecting a filtrate, adding 4.5 mol/L solution of hydrogen chloride in dioxane to the filtrate, and concentrating the solvent to obtain a crude product, then dissolving the crude product in water, performing extraction with ethyl acetate, discarding the organic phase and freeze-drying the aqueous phase to obtain the(S)-3-(2,2,2-trifluoroethyl) pyrrolidine hydrochloride;
wherein the mass ratio of(S)-3-(2,2,2-trifluoroethyl)-N—((R)-1-(naphthalen-1-yl)ethyl) pyrrolidine, methanol and 10% Pd/C catalyst was 1:5:0.5.

### Example 6

This Example is a preparation method of(S)-3-(2,2,2-trifluoroethyl) pyrrolidine hydrochloride. As shown in FIG. 3, the preparation method comprises the following steps:
(a) dissolving R-naphthylethylamine in dichloromethane, adding an aqueous sodium hydroxide solution, stirring vigorously, adding dropwise a solution of 4-chlorobutyryl chloride in dichloromethane, reacting at room temperature for 1 hour, and then raising the temperature of the reaction solution to 55° C. for further reacting for 4 hours; after the reaction was completed, separating the liquids, extracting the aqueous phase twice with dichloromethane, combining the organic phases, drying over anhydrous sodium sulfate, filtering, and concentrating the solvent to obtain the product N—(R)-(1-(naphthalen-1-yl)ethyl) pyrrolidone as a yellow oil;
wherein: the mass ratio of R-naphthylethylamine, dichloromethane, the aqueous sodium hydroxide solution and the solution of 4-chlorobutyryl chloride in dichloromethane was 1:6:1.6:0.82, wherein the concentration of the aqueous sodium hydroxide solution was 50% and the concentration of the solution of 4-chlorobutyryl chloride in dichloromethane was 50%;
(b) dissolving N—(R)-(1-(naphthalen-1-yl)ethyl) pyrrolidone in N,N-dimethylformamide and cooling the mixture to 4° C., adding trifluoroacetic anhydride, then slowly adding aluminum trichloride and stirring until it was completely dissolved to obtain a mixed solution, raising the temperature of the mixed solution to 55° C. for reaction for 30 hours, cooling the reaction solution to room temperature, adding water to quench the reaction, performing extraction with ethyl acetate for 3 times, combining the organic phases, drying over anhydrous sodium sulfate, filtering, concentrating the solvent, and performing column chromatography (petroleum ether/ethyl acetate=10:1) to obtain the product 3-(1-chloro-2,2,2-trifluoroethylidene)-N—((R)-1-(naphthalen-1-yl)ethyl) pyrrolidone as a blackish oil,
wherein the mass ratio of (R)—N-(1-(naphthalen-1-yl)ethyl) pyrrolidone, N,N-dimethylformamide, trifluoroacetic anhydride and aluminum trichloride was 1:20:3:3;
(c) dissolving 3-(1-chloro-2,2,2-trifluoroethylidene)-N—((R)-1-(naphthalen-1-yl)ethyl) pyrrolidone in methanol and adding Raney nickel as a catalyst, then reacting at room temperature under hydrogen for 20 hours, filtering the reaction solution, collecting a filtrate, concentrating the filtrate, performing extraction with ethyl acetate for 3 times, washing the product to neutral with saturated sodium bicarbonate, combining the organic phases, drying over anhydrous sodium sulfate, filtering, and concentrating the solvent to obtain the product(S)-3-(2,2,2-trifluoroethyl)-N—((R)-1-(naphthalen-1-yl)ethyl) pyrrolidone as a colorless oil,
wherein the mass ratio of 3-(1-chloro-2,2,2-trifluoroethylidene)-N—((R)-1-(naphthalen-1-yl)ethyl) pyrrolidone, methanol and the catalyst Raney nickel was 1:40:1;
(d) dissolving(S)-3-(2,2,2-trifluoroethyl)-N—((R)-1-(naphthalen-1-yl)ethyl) pyrrolidone in anhydrous tetrahydrofuran and cooling the mixture to 2° C., then adding a solution of borane in tetrahydrofuran and reacting at 65° C. for 3.5 hours, cooling the reaction solution to room temperature, adding methanol with a volume that was 5 times the volume of the reaction solution for quenching the reaction, concentrating the solvent, adding methanol with a volume that was 5 times the volume of the reaction solution again and reacting at 65° C. for 8 hours, then rotary evaporating the reaction solution to dryness and dissolving in 4 mol/L aqueous hydrochloric acid solution, performing back-extraction with ethyl acetate, discarding the organic phase, adjusting the aqueous phase to be alkaline with 6 mol/L aqueous sodium hydroxide solution, performing extraction with ethyl acetate, drying the organic phase over anhydrous sodium sulfate, filtering, and concentrating the solvent to obtain the product(S)-3-(2,2,2-trifluoroethyl)-N—((R)-1-(naphthalen-1-yl)ethyl) pyrrolidine as a light yellow oil;
wherein the mass ratio of(S)-3-(2,2,2-trifluoroethyl)-N—((R)-1-(naphthalen-1-yl)ethyl) pyrrolidone, anhydrous tetrahydrofuran and the solution of borane in tetrahydrofuran was 1:10:4; and the concentration of the solution of borane in tetrahydrofuran was 1.2 mol/L;
(e) dissolving(S)-3-(2,2,2-trifluoroethyl)-N—((R)-1-(naphthalen-1-yl)ethyl) pyrrolidine in methanol and adding 10% Pd/C as a catalyst, then reacting at room temperature under hydrogen for 15 hours, filtering the reaction solution, collecting a filtrate, adding 3.5 mol/L solution of hydrogen chloride in dioxane to the filtrate, and concentrating the solvent to obtain a crude product, then dissolving the crude product in water and performing extraction with ethyl acetate, discarding the organic phase and freeze-drying the aqueous phase to obtain the(S)-3-(2,2,2-trifluoroethyl) pyrrolidine hydrochloride;
wherein the mass ratio of(S)-3-(2,2,2-trifluoroethyl)-N—((R)-1-(naphthalen-1-yl)ethyl) pyrrolidine, methanol and 10% Pd/C catalyst was 1:20:0.3.

### Experimental Example

(S)-3-(2,2,2-trifluoroethyl) pyrrolidine hydrochloride was prepared according to examples 4-6, respectively; and the yield of the product prepared in each step of each example was calculated according to the following calculation method;
the calculation formula of the yield of reaction in each step is mₚ×Mₛ/(mₛ×Mₚ)×100%, where mₚ is the actual output of the product, Mₛ is the molar mass of the raw material, mₛ is the theoretical addition amount of the raw material, and Mₚ is the molar mass of the product.

The calculation results are as shown in Table 2:

TABLE 2

| | Exam- | Exam- | Exam- |
|---|---|---|---|
| Group/Yield | ple 1 | ple 2 | ple 3 |
| N-(R)-(1-(naphthalen-1- | 83.3% | 80.4% | 76.8% |
| yl)ethyl)pyrrolidone | | | |
| 3-(1-chloro-2,2,2-trifluoroethylidene)-N- | 79.6% | 77.5% | 70.5% |
| ((R)-1-(naphthalen-1- | | | |
| yl)ethyl)pyrrolidone | | | |
| (S)-3-(2,2,2-trifluoroethyl)-N-((R)-1- | 63.3% | 32.9% | 54.6% |
| (naphthalen-1-yl)ethyl)pyrrolidone | | | |
| (S)-3-(2,2,2-trifluoroethyl)-N-((R)-1- | 85.9% | 85.3% | 77.8% |
| (naphthalen-1-yl)ethyl)pyrrolidine | | | |
| (S)-3-(2,2,2-trifluoroethyl)pyrrolidine | 93.7% | 91.1% | 77.5% |
| hydrochloride | | | |

It can be seen from Table 2 that:
the preparation method of the present invention realizes the preparation of(S)-3-(2,2,2-trifluoroethyl) pyrrolidine hydrochloride by using R-naphthylethylamine as a raw material. By defining specific processes and parameters in the preparation method, the(S)-3-(2,2,2-trifluoroethyl) pyrrolidine hydrochloride is obtained with high yield and purity. Moreover, the preparation method has simple operation and can realize mass production.

Finally, it should be noted that the above embodiments are only used to illustrate, rather than limit, the technical solutions of the present invention. Although the present invention has been described in detail with reference to the foregoing embodiments, those having ordinary skills in the art should understand that: the technical solutions described in the foregoing embodiments can still be modified, or a part or all of the technical features therein can be equivalently substituted; and these modifications or substitutions will not make the corresponding technical solutions essentially depart from the scope of the technical solutions of the embodiments of the present invention, and shall be covered by the claims and description of the present invention.

What is claimed is:

1. A preparation method of 3-(2,2,2-trifluoroethyl) pyrrolidine hydrochloride, comprising the following steps:
(a) reacting N-benzylpyrrolidone with trifluoroacetic anhydride to obtain 3-(1-chloro-2,2,2-trifluoroethylidene)-N-benzylpyrrole;
(b) dissolving 3-(1-chloro-2,2,2-trifluoroethylidene)-N-benzylpyrrole in methanol, and carrying out a substitution reaction in the presence of a catalyst and hydrogen to obtain 3-(2,2,2-trifluoroethyl)-N-benzylpyrrolidone;
(c) reacting 3-(2,2,2-trifluoroethyl)-N-benzylpyrrolidone with borane, and adding methanol into the reaction mixture for further reaction to obtain 3-(2,2,2-trifluoroethyl)-N-benzylpyrrolidine; and
(d) dissolving 3-(2,2,2-trifluoroethyl)-N-benzylpyrrolidine in methanol, and carrying out a reaction in the presence of a catalyst and hydrogen, filtering the reaction mixture after the reaction, collecting a filtrate, adding a solution of hydrogen chloride in dioxane to the filtrate, and concentrating the solvent to obtain a crude product, then dissolving the crude product in water, performing extraction, and freeze-drying the aqueous phase to obtain the 3-(2,2,2-trifluoroethyl) pyrrolidine hydrochloride.

2. The preparation method according to claim 1, wherein in the step (a), reacting N-benzylpyrrolidone with trifluoroacetic anhydride specifically comprises:
dissolving N-benzylpyrrolidone in N,N-dimethylformamide and cooling the mixture to 3-8° C., and then adding trifluoroacetic anhydride and aluminum trichloride successively for reaction.

3. The preparation method according to claim 2, wherein in the step (a), the mass ratio of N-benzylpyrrolidone, N,N-dimethylformamide, trifluoroacetic anhydride and aluminum trichloride is 1: (1-20): (1-4): (1-3); and
the reaction temperature is 55-65° C., and the reaction time is 12-40 hours.

4. The preparation method according to claim 2, wherein the step (a) further comprises subjecting a mixture obtained after the reaction to extraction and column chromatography, wherein a solvent used for the extraction is ethyl acetate, and developing agents used for the column chromatography are PE and EA with a volume ratio of 10:1.

5. The preparation method according to claim 1, wherein in the step (b), the mass ratio of 3-(1-chloro-2,2,2-trifluoroethylidene)-N-benzylpyrrole, methanol and the catalyst is 1: (1-20): (0.01-0.50); and
the catalyst is 10% Pd/C.

6. The preparation method according to claim 1, wherein in the step (b), the conditions for the substitution reaction are as follows:
a reaction temperature at room temperature, and a reaction time of 10-15 hours;
preferably, the step (b) further comprises: filtering a mixture obtained after the reaction, collecting a filtrate, concentrating the filtrate, then performing extraction with ethyl acetate for 2-4 times, and then washing the organic phase with a saturated sodium bicarbonate aqueous solution until it is neutral, combining the organic phases, drying over anhydrous sodium sulfate, filtering, and concentrating the solvent to obtain the 3-(2,2,2-trifluoroethyl)-N-benzylpyrrolidone.

7. The preparation method according to claim 1, wherein in the step (c), reacting 3-(2,2,2-trifluoroethyl)-N-benzylpyrrolidone with borane specifically comprises:
dissolving 3-(2,2,2-trifluoroethyl)-N-benzylpyrrolidone in anhydrous tetrahydrofuran and cooling the mixture to 0-4° C., and then adding a solution of borane in tetrahydrofuran for reaction, wherein the mass ratio of 3-(2,2,2-trifluoroethyl)-N-benzylpyrrolidone, anhydrous tetrahydrofuran and the solution of borane in tetrahydrofuran is 1: (1-20): (2-10); the concentration of the solution of borane in tetrahydrofuran is 0.8-1.2 mol/L; the reaction temperature is 30-65° C., and the reaction time is 3.5-4.5 hours.

8. The preparation method according to claim 1, wherein in the step (c), adding methanol into the reaction mixture for further reaction specifically comprises:
after cooling the reaction mixture, adding methanol with a volume that is 0.1-10 times the volume of the reaction mixture for quenching, concentrating the solvent, and then adding methanol with a volume that is 1-10 times the volume of the reaction mixture for reaction, wherein the reaction temperature is 65-75° C. and the reaction time is 1-8 hours;
preferably, the step (c) further comprises: concentrating the mixture obtained after the reaction, then dissolving it in an aqueous hydrochloric acid solution, performing extraction with ethyl acetate, discarding the organic phase, adjusting the aqueous phase to be alkaline, and performing extraction with ethyl acetate for another 2-4 times, then combining the organic phases, drying over anhydrous sodium sulfate, filtering, and concentrating the solvent to obtain the 3-(2,2,2-trifluoroethyl)-N-benzylpyrrolidine; wherein the concentration of the aqueous hydrochloric acid solution is 3-5 mol/L.

9. The preparation method according to claim 1, wherein in the step (d), the mass ratio of 3-(2,2,2-trifluoroethyl)-N-benzylpyrrolidine, methanol and the catalyst is 1: (1-20): (0.01-0.50); and
the catalyst is 10% Pd/C.

10. The preparation method according to claim 1, wherein in the step (d), the reaction conditions are as follows: a reaction temperature at room temperature and a reaction time of 10-30 hours; and
a mass ratio of the solution of hydrogen chloride in dioxane to the filtrate being 1:0.10-2;
and a concentration of the solution of hydrogen chloride in dioxane being 3.5-4.5 mol/L.

11. A preparation method of(S)-3-(2,2,2-trifluoroethyl) pyrrolidine hydrochloride, comprising the following steps:
(a) reacting R-naphthylethylamine with 4-chlorobutyryl chloride to obtain N—(R)-(1-(naphthalen-1-yl)ethyl) pyrrolidone;
(b) reacting N—(R)-(1-(naphthalen-1-yl)ethyl) pyrrolidone with trifluoroacetic anhydride to obtain 3-(1-chloro-2,2,2-trifluoroethylidene)-N—((R)-1-(naphthalen-1-yl)ethyl) pyrrolidone;
(c) dissolving 3-(1-chloro-2,2,2-trifluoroethylidene)-N—((R)-1-(naphthalen-1-yl)ethyl) pyrrolidone in methanol, and carrying out a substitution reaction in the presence of a catalyst and hydrogen to obtain(S)-3-(2,2,2-trifluoroethyl)-N—((R)-1-(naphthalen-1-yl)ethyl) pyrrolidone;
(d) reacting(S)-3-(2,2,2-trifluoroethyl)-N—((R)-1-(naphthalen-1-yl)ethyl) pyrrolidone with borane, and adding methanol into the reaction mixture for further reaction to obtain(S)-3-(2,2,2-trifluoroethyl)-N—((R)-1-(naphthalen-1-yl)ethyl) pyrrolidine; and
(e) dissolving(S)-3-(2,2,2-trifluoroethyl)-N—((R)-1-(naphthalen-1-yl)ethyl) pyrrolidine in methanol and carrying out a reaction in the presence of a catalyst and hydrogen, filtering the reaction mixture after the reaction, collecting a filtrate, adding a solution of hydrogen chloride in dioxane to the filtrate, and concentrating the solvent to obtain a crude product, then dissolving the crude product in water, performing extraction with ethyl acetate, and freeze-drying the aqueous phase to obtain the(S)-3-(2,2,2-trifluoroethyl) pyrrolidine hydrochloride.

12. The preparation method according to claim 11, wherein in the step (b), reacting N—(R)-(1-(naphthalen-1-yl)ethyl) pyrrolidone with trifluoroacetic anhydride specifically comprises:
dissolving N—(R)-(1-(naphthalen-1-yl)ethyl) pyrrolidone in N,N-dimethylformamide and cooling the mixture to 3-8° C., and then adding trifluoroacetic anhydride and aluminum trichloride successively for reaction.

13. The preparation method according to claim 12, wherein in the step (b), the mass ratio of N—(R)-(1-(naphthalen-1-yl)ethyl) pyrrolidone, N,N-dimethylformamide, trifluoroacetic anhydride and aluminum trichloride is 1: (1-20): (1-4): (0.3-3); and
the reaction temperature is 55-65° C., and the reaction time is 12-40 hours.

14. The preparation method according to claim 12, wherein the step (b) further comprises subjecting a mixture obtained after the reaction to extraction and column chromatography, wherein a solvent used for the extraction is ethyl acetate, and developing agents used for the column chromatography are PE and EA with a volume ratio of 10:1.

15. The preparation method according to claim 11, wherein in the step (c), the mass ratio of 3-(1-chloro-2,2,2-trifluoroethylidene)-N—((R)-1-(naphthalen-1-yl)ethyl) pyrrolidone, methanol and the catalyst is 1: (1-50): (0.1-1); and
the catalyst is Raney nickel.

16. The preparation method according to claim 11, wherein in the step (c), the conditions for the substitution reaction are as follows:
a reaction temperature at room temperature and a reaction time of 20-24 hours;
preferably, the step (c) further comprises: filtering a mixture obtained after the reaction, collecting a filtrate, concentrating the filtrate, then performing extraction with ethyl acetate for 2-4 times, combining the organic phases, drying over anhydrous sodium sulfate, filtering, and concentrating the solvent to obtain the(S)-3-(2,2,2-trifluoroethyl)-N—((R)-1-(naphthalen-1-yl)ethyl) pyrrolidone.

17. The preparation method according to claim 11, wherein in the step (d), reacting(S)-3-(2,2,2-trifluoroethyl)-N—((R)-1-(naphthalen-1-yl)ethyl) pyrrolidone with borane specifically comprises:
dissolving(S)-3-(2,2,2-trifluoroethyl)-N—((R)-1-(naphthalen-1-yl)ethyl) pyrrolidone in anhydrous tetrahydrofuran and cooling the mixture to 0-4° C., and then adding a solution of borane in tetrahydrofuran for reaction, wherein the mass ratio of(S)-3-(2,2,2-trifluoroethyl)-N—((R)-1-(naphthalen-1-yl)ethyl) pyrrolidone, anhydrous tetrahydrofuran and the solution of borane in tetrahydrofuran is 1: (1-20): (2-10); the concentration of the solution of borane in tetrahydrofuran is 0.8-1.2 mol/L; the reaction temperature is 30-65° C., and the reaction time is 3.5-4.5 hours.

18. The preparation method according to claim 11, wherein in the step (d), adding methanol into the reaction mixture for further reaction specifically comprises:
after cooling the reaction mixture, adding methanol with a volume that is 0.1-10 times the volume of the reaction mixture for quenching, concentrating the solvent, and then adding methanol with a volume that is 1-10 times the volume of the reaction mixture for reaction, wherein the reaction temperature is 65-75° C. and the reaction time is 1-8 hours;
preferably, the step (d) further comprises: concentrating the mixture obtained after the reaction, then dissolving it in an aqueous hydrochloric acid solution, performing extraction with ethyl acetate, discarding the organic phase, adjusting the aqueous phase to be alkaline, and performing extraction with ethyl acetate for another 2-4 times, then combining the organic phases and drying over anhydrous sodium sulfate, filtering, and concentrating the solvent to obtain the(S)-3-(2,2,2-trifluoroethyl)-N—((R)-1-(naphthalen-1-yl)ethyl) pyrrolidine; wherein the concentration of the aqueous hydrochloric acid solution is 3-5 mol/L.

19. The preparation method according to claim 11, wherein in the step (e), the mass ratio of(S)-3-(2,2,2-trifluoroethyl)-N—((R)-1-(naphthalen-1-yl)ethyl) pyrrolidine, methanol and the catalyst is 1: (1-20): (0.01-0.50); and
the catalyst is 10% Pd/C.

20. The preparation method according to claim 11, wherein in the step (e), the reaction conditions are as follows: a reaction temperature at room temperature and a reaction time of 10-30 hours; and
a mass ratio of the solution of hydrogen chloride in dioxane to the filtrate being 1:(0.10-2);
and a concentration of the solution of hydrogen chloride in dioxane being 3.5-4.5 mol/L.
